(19) United States
(12) Patent Application Publication
(10) Pub. No.: US 2003/0060749 A1
(43) Pub. Date: Mar. 27, 2003

(54) Pre-filled syringe and method for making same

(76) Inventor: Antoine Aneas, Menetrol (FR)

Correspondence Address: Monique A Morneault, Wallenstein & Wagner, 311 South Wacker Drive 5300, Chicago, IL, 60606, US

(21) Appl. No.: 10/149,363

(22) PCT Filed: Dec. 7, 2000
(86) PCT No.: PCT/FR00/03438

(30) Foreign Application Priority Data
Dec. 14, 1999 (FR) 99/15778

(51) Int. Cl. A61N 1/30

(52) U.S. Cl. 604/20

(57) ABSTRACT

The invention concerns a syringe comprising a syringe body (2) including an elongate barrel (4) containing a volume of medical fluid (44), one first end forming a hub (16), designed to co-operate with a needle of said syringe, a second end (6) opposite the hub and a plunger (28) mobile relative to said hub. The second end forms a closed base (6) and the plunger (28) is entirely housed inside the barrel (4). Said syringe body is inexpensive, easy to produce, provides satisfactory fluid-tightness and prevents all microbial migration towards the medical fluid.

The present invention relates to a pre-filled syringe body, to a pre-filled syringe provided with such a body, to a set of these bodies, and to a process for manufacturing such a body.

A pre-filled syringe body usually contains a volume of medicinal liquid, which is for example an injectable preparation or a charge of such a preparation. This body comprises an elongated envelope, a first end, forming end piece, and a second end opposite said end piece. Furthermore, the body slidingly receives a piston mobile with respect to the end piece.

As is known, this body can be made by injection of plastics material, an opening being arranged in its second end. The medicinal liquid is then poured through said opening and the piston is connected near the liquid thus transferred. During these operations, and during the possible storage of the syringe body, the end piece is provided with a sealing cap.

Immediately before use, the sealing cap is removed from the end piece and a connection provided with a needle is fitted on the latter. Finally, the piston is slid into the envelope, by pressure on the actuation end, so as to inject the medicinal liquid.

This known syringe body presents certain drawbacks, in particular in terms of safety of use. In effect, the medicinal liquid can flow through the opening made in the bottom of the body, particularly if the piston rod is pulled too far to the rear.

In addition, this syringe body does not satisfactorily avoid microbial migration towards the medicinal liquid that it contains. In effect, microbes can reach this liquid through the opening of the second end of the body, both during the phases of manufacture and of storage, and during actual use of the syringe body. Such migration may in addition be initiated from the actuation end of the body located outside the envelope and which may therefore be contaminated.

Finally, this syringe body involves relatively long and complex phases of production and its cost price is high.

In order to overcome these different drawbacks, the invention proposes a pre-filled syringe body which is easy and inexpensive to manufacture, which guarantees a reliable conservation of the medicinal liquid that it contains, even in the case of long-term storage, and which avoids, to a large extent, any risk of microbial contamination during the phases of storage and use.

To that end, it relates to a pre-filled syringe body containing a volume of medicinal liquid, said body comprising an elongated envelope, a first end forming end piece, intended to cooperate with a needle of said syringe, a second end opposite said end piece, and a piston mobile with respect to said end piece, characterized in that said second end forms a closed bottom, integral with the envelope, and said piston is housed entirely within said body.

The syringe body according to the invention ensures a satisfactory seal, being given that it presents a closed bottom. This therefore avoids leakages of liquid flowing via the opening provided in the prior art, in the case of poor manipulation. This closed bottom also protects from any microbial contamination.

Furthermore, this bottom is integral with the envelope. This is advantageous in terms of simplicity of manufacture and of seal.

Finally, being given that the piston is housed entirely within the envelope, the actuation end of the piston cannot come into contact with a non-sterile body or member, this avoiding any microbial contamination via this actuation end.

According to an advantageous characteristic of the invention, the envelope comprises an actuation portion, deformable in the direction of displacement of the piston, which terminates in a region for drive of an actuation end of the piston.

This allows the piston to move easily during the phase of injection of the medicinal liquid.

According to other characteristics of the invention:

the drive region comprises the bottom of said body, said bottom being fast with the actuation end, in particular by welding;

said deformable portion is in the form of bellows.

According to an advantageous characteristic of the invention, said deformable portion extends, opposite the bottom, in a portion for receiving the volume of medicinal liquid, and said deformable portion is provided with a zone for abutment of a user's fingers, adjacent said receiving portion.

This zone of abutment allows a user to abut two of his/her fingers, for example the fore-finger and the middle finger, then to easily exert a pressure with his/her thumb on the closed bottom of the body, which then drives the actuation end of the piston.

According to another advantageous characteristic of the invention, the end piece is provided with a removable sealing member integral with said envelope.

This member provides the end piece with a satisfactory tightness during storage of the syringe body. Furthermore, being given that it is integral, this member cannot be fitted on the end piece again after having been removed therefrom, which avoids any problem of microbial contamination. This is to be compared with the prior art which employs sealing caps which can be repositioned on the end piece after having been removed therefrom.

According to another characteristic of the invention, the sealing member is breakable, by thinning of material.

According to another characteristic of the invention, the sealing member is fast with gripping means, in particular a tongue. This renders use particularly simple and allows the cap to be easily detached.

According to another advantageous characteristic of the invention, the end of said piston in contact with the liquid, opposite the actuation end, is arranged, in a transversely compressed state, in a sleeve fast with the envelope, a lubricant being interposed between the facing walls of this contact end and the sleeve.

This guarantees an easy slide, in service, of the contact end with respect to the inner walls of the envelope. Moreover, being given that this end is compressed in the sleeve, it can be radially deformed towards the outside so as to cooperate intimately with the walls of the envelope in order to ensure satisfactory tightness.

According to another characteristic of the invention, the inner transverse dimensions of the sleeve are smaller than those of the envelope.

The invention also relates to a pre-filled syringe comprising a body and a needle fast with said body, characterized in that the body is such as defined hereinabove.

The invention also relates to a set of a plurality of pre-filled syringe bodies such as defined hereinabove, these bodies being removably joined in two's.

This is advantageous in terms of storage and, in addition, renders use simple. A sheet of syringe bodies can consequently be manufactured, from which each body is detached immediately before it is used.

According to another characteristic of the invention, two adjacent bodies are joined by welding of the facing ends of their deformable portions and/or of their gripping tongues.

Finally, the invention relates to a process for manufacturing a pre-filled syringe body containing a volume of medicinal liquid, said body comprising an elongated envelope, a first-end forming end piece, intended to cooperate with a needle of said syringe, a second end opposite said end piece and a piston mobile with respect to said end piece, characterized in that it comprises the following steps of:

making a blank of said body, with said first end closed;

pouring said liquid in the inner volume of the blank;

connecting the piston in the vicinity of the liquid;

making said second end in the form of a closed bottom.

The process of manufacture of the invention guarantees a substantial simplification, as well as a noteworthy reduction of costs, over the prior art.

In effect, according to the invention, the liquid is poured and the piston is connected before the bottom of the body is made, this allowing particularly rapid manufacture. This is to be compared with the prior art in which, once the body is made, the liquid is transferred then the elements constituting the piston connected, via the orifice made in the bottom.

According to an advantageous characteristic of the invention, the closed bottom is made while making a deformable portion of said envelope.

This is advantageous in terms of rapidity and simplicity of manufacture.

According to other characteristics of the invention:

said deformable portion is made by connecting, perpendicularly to the principal axis of the envelope, at least two mould elements provided with notched moulding walls;

said piston is connected after having surrounded it, in a transversely compressed state, by means of a sleeve, the outer walls of said sleeve are brought near the facing inner walls of the envelope, and the sleeve is connected with the envelope;

the outer walls of the sleeve are brought near the inner walls of the envelope, these inner walls being in a transversely expanded state, and these inner walls are shrunk, particularly by cooling, so as to connect the sleeve and the envelope;

the body is made by blowing the plastics material constituting it.

The invention will be more readily understood on reading the following description given by way of example with reference to the accompanying drawings, in which:

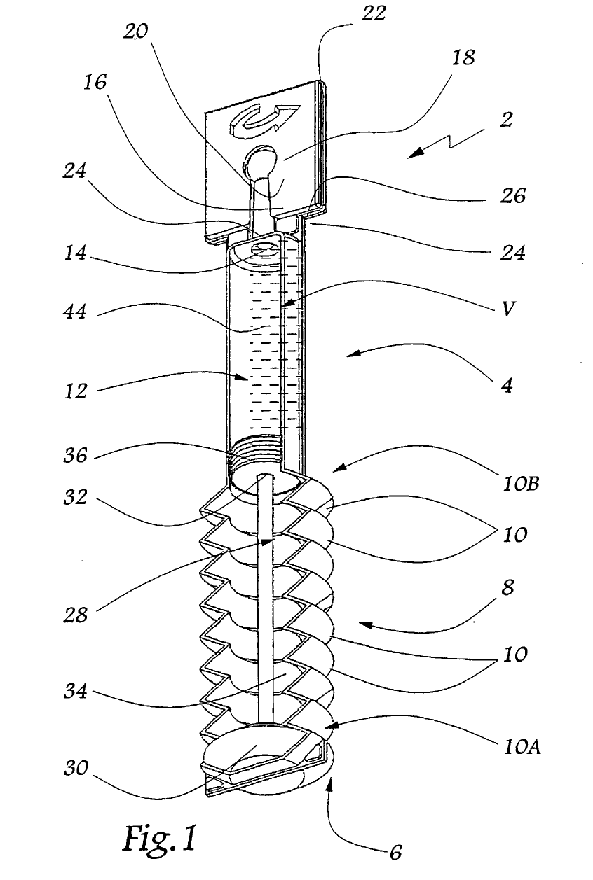

FIG. 1 is a view in perspective, with parts torn away, of a syringe body according to the invention.

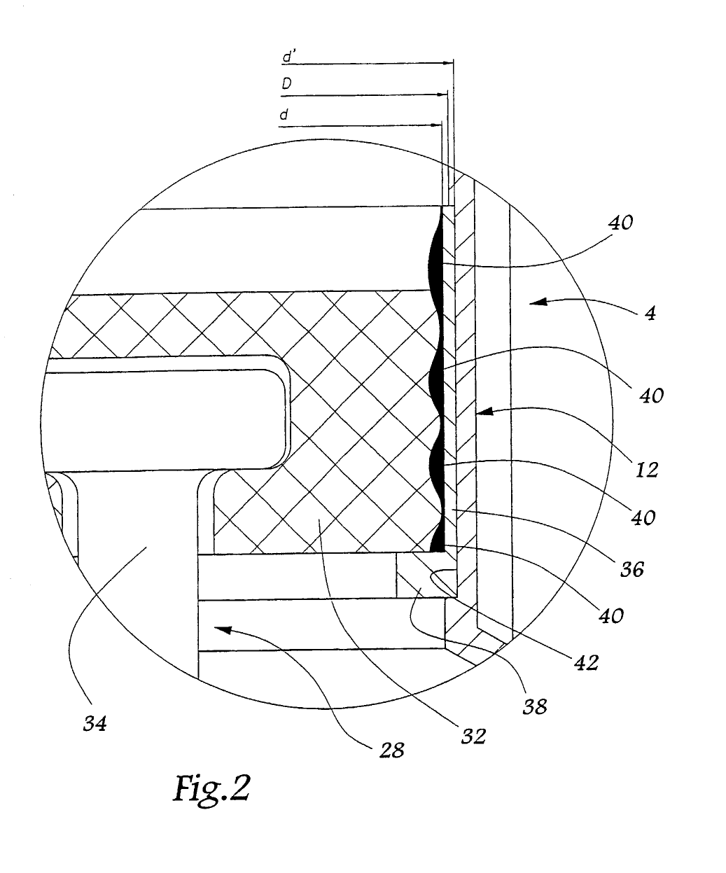

FIG. 2 is a view in longitudinal section, on a larger scale, illustrating the relative positioning of the envelope and the thrust end of the piston belonging to the syringe body of FIG. 1.

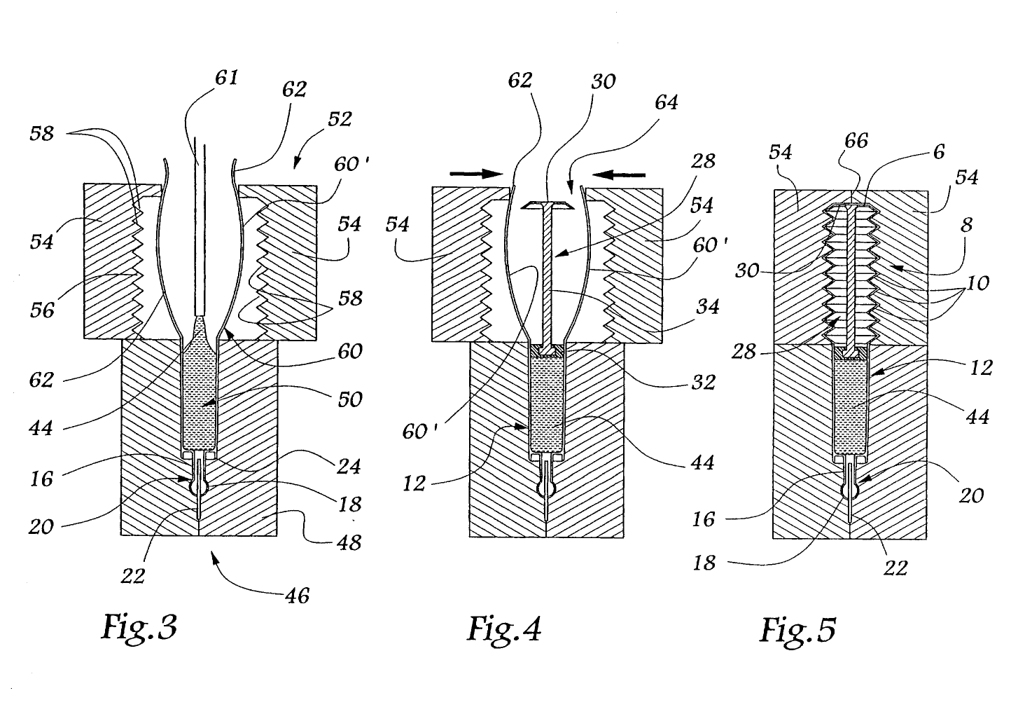

FIGS. 3 to 5 are views in section illustrating three successive phases of manufacture of the syringe body of FIG. 1.

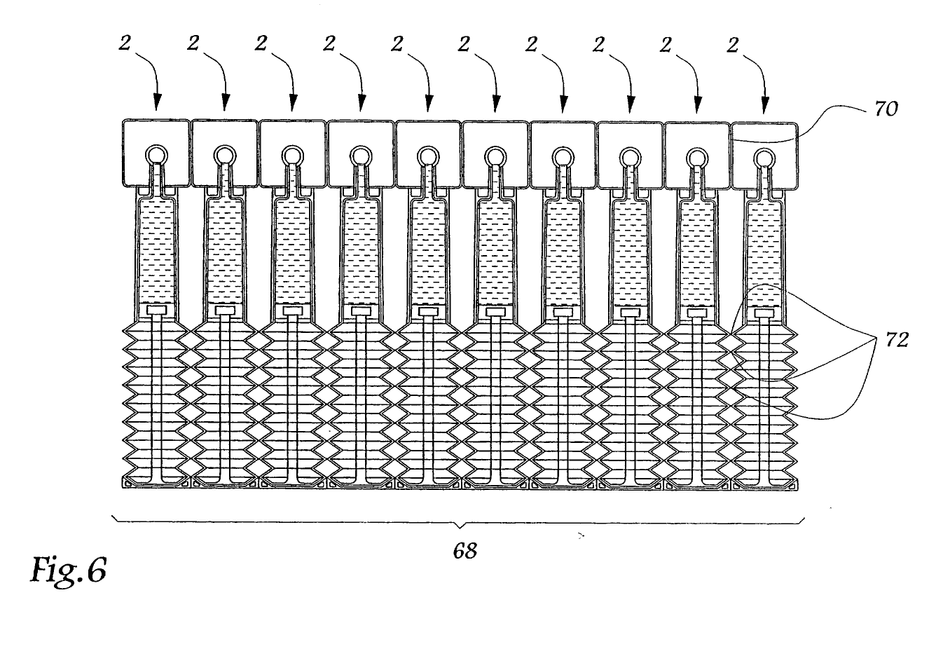

FIG. 6 is a side view illustrating a set of a plurality of syringe bodies, of which each is identical to that of FIG. 1.

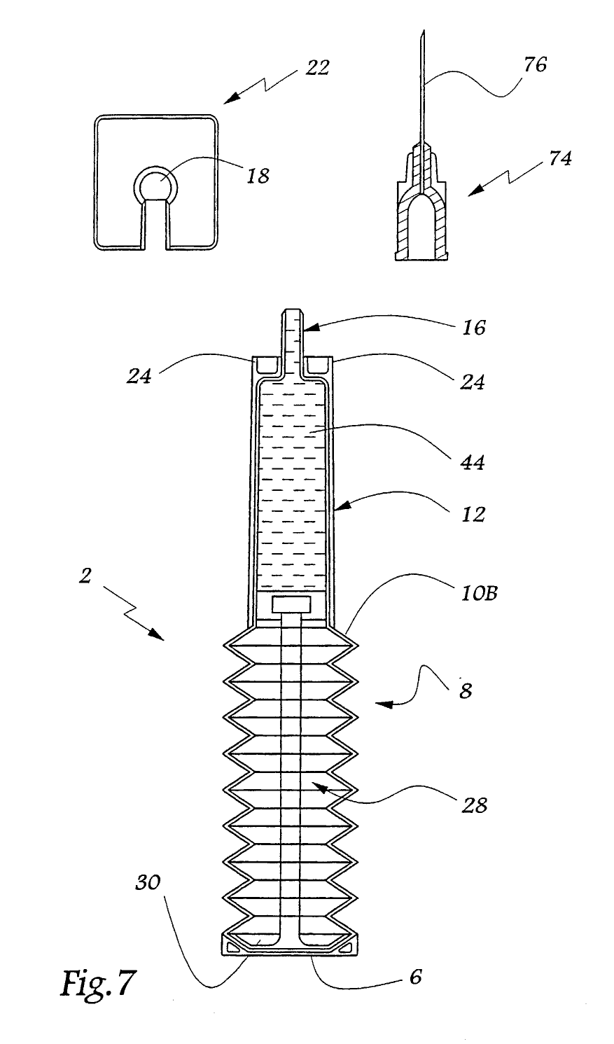

FIG. 7 is a side view, illustrating the use of the syringe body of FIG. 1.

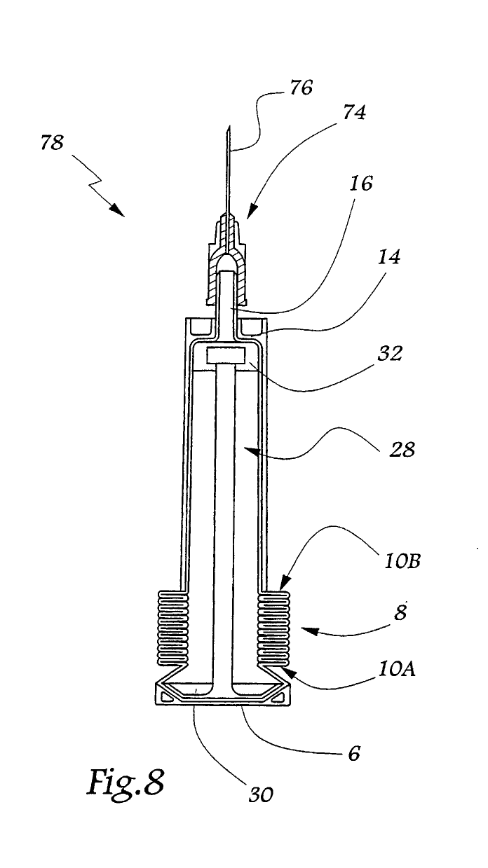

FIG. 8 is a view similar to FIG. 7, illustrating a syringe according to the invention in its final phase of use, and

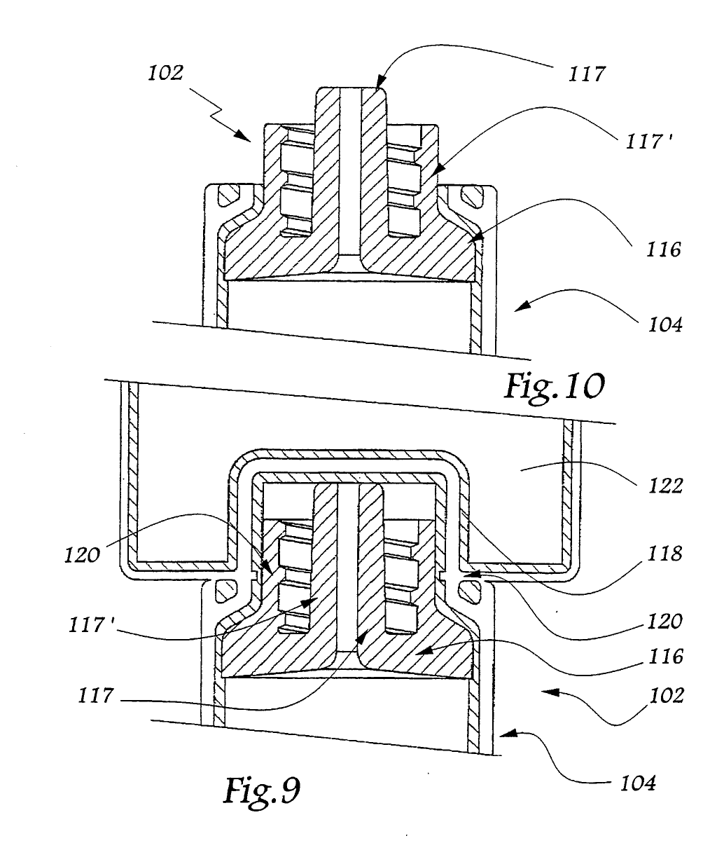

FIGS. 9 and 10 are views in section, on a larger scale, illustrating the end piece of a syringe body according to a variant of the invention, in two successive phases of its use.

The syringe body 2 shown in FIG. 1, which is made of a plastics material such as polyethylene, comprises a hollow elongated envelope 4 terminating in a closed bottom 6 of the body, integral with this envelope. The latter is provided with a bellows-type element 8 which constitutes a deformable portion of this envelope 4, and defines a plurality of notches or corrugations 10.

The envelope 4 also comprises a cylindrical portion 12 whose diameter corresponds substantially to that of the inner part of the notches 10. This cylindrical portion 12 terminates, opposite the bottom 6, in a re-entrant radial shoulder 14 from which a tubular end piece 16, coaxial to the envelope 4, extends. This end piece is fast with a sealing member 18 with closed bottom. The latter, which is integral with the end piece 16, is substantially spherical. This end piece 16 and the member 18 are joined by an annular breakable zone 20, formed by thinning of the constituent material.

The cap 18 is fast with a tongue 22 for gripping, which is connected to the shoulder 14 via bridges 24. The shoulder 14 and the bridges 24 are connected by two breakable zones 26 formed by thinning of the constituent material.

The syringe body 2 also comprises a piston, made of plastics material, which is generally designated by reference 28. This piston comprises an actuation end 30 whose shape is complementary of that of the inner walls of the notch 10A adjacent the bottom 6. This actuation end 30 is connected to the inner wall of the bottom 6 by welding, as will be explained hereinbelow. The piston 28 also comprises an end 32, or core, intended to be in contact with a medicinal liquid. This core 32 is connected to the actuation end 30 by a rod 34, coaxial to envelope 4 and extending along the bellows element 8.

As is shown more precisely in FIG. 2, the outer periphery of the core 32 is surrounded by a sleeve 36, fast with the envelope 4 as will be explained hereinbelow. This sleeve 36 extends axially, in the direction of the end piece, beyond the core 32. Furthermore, it is provided, opposite this end piece, with a re-entrant flange 38 partially covering the end of the core 32 thereopposite.

The periphery of this core is corrugated, with the result that it forms, with the adjacent walls of the sleeve 36, grooves filled with a lubricant 40 such as silicone. This sleeve 36 extends in an annular recess 42 with which the inner wall of the cylindrical portion 12 is provided. At its edge oriented towards the end piece 16, this sleeve presents an inner diameter d smaller than that D of the envelope 4, but an outer diameter d′ larger than the inner diameter of this envelope.

The end piece 16 defines, with the face of the core 32 thereopposite, a useful inner volume V which is filled with a medicinal liquid 44, for example an injectable preparation or a charge for such a preparation.

The process for manufacturing the syringe body 2 will be explained with reference to FIGS. 3 to 5.

For such manufacture, a mould 46 is employed, comprising a fixed lower part 48 defining a moulding cavity 50 for forming the cylindrical portion 12, the end piece 16, the cap 18 and the tongue 22. This mould 46 also comprises a mobile part 52, formed by two mould elements 54 capable of moving with respect to each other in a direction perpendicular to the principal direction of the envelope 4. These mobile elements 54 present internal moulding walls 56 provided with notches 58 corresponding to the notches 10 of the body 2 once made.

In a first phase of manufacture, the mobile elements 54 are moved apart from each other, then a preform 60 made of plastics material is introduced into the cavity 50 and deformed by blowing in a sterile atmosphere, after having previously softened it, for example by action of heat.

The cylindrical portion 12, the end piece 16, the cap 18 and the tongue 22 are moulded in the cavity 50. Outside the mould, the core 32 is surrounded by its sleeve 36, lubricant 40 being introduced between the facing walls of this core 32 and this sleeve 36.

Still in a sterile atmosphere, the medicinal liquid 44 is then poured into the cylindrical portion 12 via a conduit 61. The edges 62 of the preform, opposite cavity 50, surround, at their upper end (not shown), this conduit 61 admitting liquid 44. The conduit 61 is then withdrawn and the upper end of the edges 62 is removed so as to produce an open end 64 via which the piston 28, provided with its sleeve (not shown), is connected above the transferred liquid 44.

Being given that the preform 60 is softened under the effect of heat, it is subjected to a slight radial expansion, with the result that the outer walls of the sleeve are free of contact with the inner walls of the preform 60 intended to form the cylindrical portion 12.

Once the piston is connected, the mobile elements 54 are brought together (FIGS. 4 and 5), with the result that their ends opposite the fixed part 48 form a mould joint 66. Such bringing together forms the bottom 6 of the body and the bellows 8 of the envelope 4, the lateral edges 60′ of the preform 60 being connected by a line of weld. Furthermore, the notches 58 of the moulding walls of elements 54 produce the notches 10 of the bellows element 8.

The plastics material constituting the envelope 4 is then cooled, which provokes a slight reduction of the transverse dimensions of this envelope, and in particular of the cylindrical portion 12. Consequently, the sleeve 36, whose transverse dimensions do not vary, is rendered fast with the facing zone of the cylindrical portion 12, which shrinks. Furthermore, a phenomenon of weld occurs between the facing walls of the bottom 6 and of the actuation end 30 of the piston 28.

FIGS. 3 to 5 describe the formation of a single body 2. However, it is possible to produce a plurality of such bodies, side by side, by means of a single blowing operation and a single mould. This then leads to obtaining a set 68 formed by a plurality of syringe bodies 2, of which the adjacent regions are mutually connected by welding. More precisely, the adjacent walls of the tongues 22 are joined by a line of weld 70, while the adjacent zones of the bellows element 8 are joined by a plurality of welding spots 72 arranged along the welds joining the lateral edges 60′ of the preform 60.

Functioning of the syringe body 2 will now be explained, with reference to FIGS. 7 and 8. Firstly, a body 2 is detached from the set 68, shown in FIG. 6, by action on the line 70 and the welding spots 72. Then the tongue 22 is detached, for example by rotating it about the principal axis of the envelope 4.

On the end piece 16 whose end has been opened, there is then fitted a connector 74 of known type, provided with an injection needle 76, so as to form a syringe 78 (FIG. 8). The operator then places two of his/her fingers, for example the fore finger and the middle finger, on the notch 10B of the bellows, adjacent the cylindrical portion 12. Once his two fingers are in abutment on the notch 10B, the operator presses with his thumb on the bottom 6, this contributing to retracting the notches 10 of the bellows element.

This action induces the displacement of the core 32, with respect to the sleeve (not shown in FIGS. 7 and 8), in the direction of the end piece. Such displacement is facilitated by the presence of the lubricant 40. The medicinal liquid 44 is thus injected into a patient via the needle 76. At the end of the operation, the bellows element 8 is entirely retracted and the thrust core 32 of the piston 28 is in abutment against the inner wall of the shoulder 14, as illustrated in FIG. 8.

It may also be envisaged to pour the medicinal liquid 44 into an annexed flask containing an additive such as a powder or a lyophilisate. This operation may be carried out via a connection device such as the one described in WO-A-97/10156. The medicinal liquid and its additive are then mixed inside the flask and the mixture thus formed is transferred into the envelope 4, having previously returned the bellows element into its expanded position illustrated in FIG. 7. This mixture may then be injected into the patient in accordance with the procedure described hereinabove.

FIGS. 9 and 10 show a variant embodiment of the invention in which the envelope 104 is extended by a cap 118, forming a sealing member, which is integral therewith via an annular breakable zone 120. Furthermore, this cap 118 is fast with a gripping tongue 122.

An end piece 116 is connected in the vicinity of the end of the envelope adjacent the cap 118, one end of this end piece being in abutment against the facing wall of the cap 118. This end piece 116, which has an opening channel 117, coaxial to the envelope. hollowed out therein, is provided with a male end 117′ of the “LUER LOCK” type allowing a female end (not shown) of “LUER LOCK” type to be fitted in known manner.

The use of this syringe body 102 consists, similarly to what has been described hereinbefore, in rotating the tongue 122 so as to detach it from the envelope 104, the channel 117′ then placing the inner volume of the envelope 104 in communication with the outside. The threaded connector mentioned above is then fitted on the tapping 117 and the medicinal liquid is injected.

1. Body (2; 102) of pre-filled syringe (78), containing a volume of medicinal liquid (44), said body comprising an elongated envelope (4; 104), a first end forming end piece (16; 116), intended to cooperate with a needle (76) of said syringe (78), a second end (6) opposite said end piece, and a piston (28) mobile with respect to said end piece, characterized in that said second end forms a closed bottom (6), integral with the envelope (4; 104), and said piston (28) is housed entirely within said body (2; 102).

2. Syringe body according to claim 1, characterized in that said envelope (4; 104) comprises an actuation portion (8) deformable in the direction of displacement of the piston (28), which terminates in a region (6) for drive of an actuation end (30) of the piston.

3. Syringe body according to claim 2, characterized in that the drive region comprises the bottom (6) of said body (2; 102), said bottom being fast with the actuation end (30), in particular by welding.

4. Syringe body according to one of claims 2 or 3, characterized in that said deformable portion is a bellows-type element (8).

5. Syringe body according to one of claims 2 to 4, characterized in that said deformable portion (8) extends, opposite the bottom (6), in a portion (12) for receiving the volume of medicinal liquid (44) and in that said deformable portion (8) is provided with a zone of abutment (10B) for a user's fingers, adjacent said receiving portion (2).

6. Syringe body according to one of claims 1 to 5, characterized in that said end piece (16; 116) is provided with a removable sealing member (18; 118) integral with said envelope.

7. Syringe body according to claim 6, characterized in that said sealing member (18; 118) is breakable, by thinning of material (20; 120).

8. Syringe body according to one of claims 6 or 7, characterized in that said sealing member (18; 118) is fast with gripping means, in particular a tongue (22; 122).

9. Syringe body according to one of claims 1 to 8, characterized in that the end (32) of said piston (28) in contact with the liquid, opposite the actuation end (30), is arranged, in a transversely compressed state, in a sleeve (36) fast with the envelope (4; 104), a lubricant (40) being interposed between the facing walls of this contact end (32) and the sleeve (36).

10. Syringe body according to claim 9, characterized in that the inner transverse dimensions (d) of the sleeve (36) are smaller than those (D) of the envelope (4; 104).

11. Pre-filled syringe (78) comprising a body (2) and a needle (76) fast with said body, characterized in that said body (2) is in accordance with any one of the preceding Claims.

12. Set (68) of a plurality of pre-filled syringe bodies (2) according to any one of claims 1 to 10, said bodies (2) being removably joined in two's.

13. Set according to claim 12, in combination with one of claims 8 to 10, characterized in that two adjacent bodies are joined by welding of the facing ends of their deformable portions (8) and/or of their gripping tongues (22).

14. Process for manufacturing a body (2; 102) of a pre-filled syringe, containing a volume of medicinal liquid (44), said body comprising an elongated envelope (4; 104), a first end forming end piece (16; 116), intended to cooperate with a needle of said syringe, a second end (6) opposite said end piece, and a piston (28) mobile with respect to said end piece, characterized in that it comprises the following steps of:
making a blank (60) of said body, with said first end (18) closed:
pouring said liquid (44) in the inner volume of the blank (60);
connecting the piston (28) in the vicinity of the liquid (44):
making said second end in the form of a closed bottom (6).

15. Process of manufacture according to claim 14, characterized in that said closed bottom (6) is made while producing a deformable portion (8) of said envelope (4).

16. Process of manufacture according to claim 15, characterized in that said deformable portion (8) is made by connecting at least two mould elements (54) provided with notched moulding walls (56), perpendicularly to the principal axis of the envelope (4).

17. Process of manufacture according to one of claims 14 to 16, characterized in that said piston (28) is connected after having surrounded it, in a transversely compressed state, by means of a sleeve (36), the outer walls of said sleeve (36) are brought near the facing inner walls of the envelope (4), and the sleeve (36) is connected with the envelope (4).

18. Process of manufacture according to claim 17, characterized in that the outer walls of the sleeve (36) are brought near the inner walls of the envelope (4), these inner walls being in a transversely expanded state, and in that these inner walls are shrunk, particularly by cooling, so as to connect the sleeve (36) and the envelope (4).

19. Process according to any one of claims 14 to 18, characterized in that the body (2; 102) is made by blowing the plastics material constituting it.
